(19) United States
(12) Patent Application Publication
(10) Pub. No.: US 2006/0241564 A1
(43) Pub. Date: Oct. 26, 2006

(54) Steerable catheter assembly

(75) Inventors: Neil Corcoran, County Cavan (IE); Dermot O'Reilly, County Kerry (IE)

Correspondence Address: KUSNER & JAFFE;HIGHLAND PLACE SUITE 310, 6151 WILSON MILLS ROAD, HIGHLAND HEIGHTS, OH, 44143, US

(73) Assignee: Creganna Technologies Limited

(21) Appl. No.: 11/398,890

(22) Filed: Apr. 6, 2006

(30) Foreign Application Priority Data
Apr. 7, 2005 (EP) 05075800.2

(51) Int. Cl. A61M 25/00

(52) U.S. Cl. 604/523

(57) ABSTRACT

The present invention relates to a steerable catheter assembly, comprising an elongate outer shaft having a lumen therethrough and having a proximal end and a distal end; an elongate inner shaft coaxially disposed within the lumen of the outer shaft and having a proximal end and a distal end; and characterised in that at least one of the inner shaft and the outer shaft is formed with a curvature at a distal portion, such that in an aligned configuration, a distal end of the catheter assembly is disposed at a maximum deflection angle α to a longitudinal axis of the catheter assembly; and the inner shaft and the outer shaft are rotatable relative to one another out of the aligned configuration such that each shaft exerts a deflection force on the other shaft to deflect the distal end of the catheter assembly between α and 0 degrees to the longitudinal axis of the catheter assembly.

### FIELD OF THE INVENTION

The invention relates to steerable catheters for use in minimally invasive surgical procedures.

### BACKGROUND TO THE INVENTION

Catheters for insertion into bodily lumens, e.g. intravascular catheters and the like are well known in the art. Catheters typically employ elongate flexible tubes made from a synthetic plastics material or from stainless steel. Desirable characteristics of catheter tubing include “pushability”, that is the ability to transfer forces from the proximal to the distal end of the catheter. It is also an advantage for the catheter to have good “trackability”, i.e. to be sufficiently flexible as to be capable of navigating tortuous paths within a body lumen without kinking. It is also desirable for the catheter to have good “torqueability”, as manipulation of a device within a body lumen often requires small precise amounts of torque to be applied.

Non-steerable delivery devices such as hypotubes are widely used for delivery of devices in a variety of minimally invasive surgical procedures. Some examples of typical devices that may be delivered to a pre-determined area are stents and balloons. Stents may be used to hold an artery wall open where a constriction or stenosis has occurred. Repeated inflation and deflation of balloons may be used to detach plaque from artery walls. The term hypotube refers generally to a metallic tube or shaft (although plastics may also be used), preferably of stainless steel, having a lumen extending therethrough. The tube or shaft is preferably of thin-walled construction.

Metals are generally used as they provide several advantages to the surgeon when he or she delivers a device or treatment percutaneously, to locations within any lumen of the human body, including the vasculature, the bilary system, oesophagus, and gastrointestinal tract. A first advantage is the degree of pushability of the hypotube through the vasculature of the human body. Pushability may be defined as the ability to transfer energy from one end of the hypotube shaft to the other. Pushability is therefore a measure of the transfer of the force from the proximal end to the distal end of the hypotube, that is, the ratio of force applied at the proximal end by the physician as compared to the force measured at the distal end. Good pushability means that force is efficiently and effectively transferred through the hypotube.

Another advantage is the torque or torqueability provided by the hypotube. Torqueability may be defined as the ability to transmit a rotational displacement along the length of the hypotube shaft. Perfect torqueability means that one turn at the proximal end of the hypotube would result in one turn at the distal end—this is often represented as 1:1 torque.

A further advantage is the kink resistance provided by metal hypotubes. Kink resistance may be defined as the shaft's ability to maintain its cross sectional profile during deformation.

Hypotubes also provide a usable inner lumen, which may be used for delivery of inflation fluids to catheter balloons, delivery of contrast media to a site within the human body, delivery of drugs for localised treatment and a range of other applications. Furthermore, hypotubes are capable of producing exceptionally low profiles to tight tolerances. This ensures that the procedure is a comfortable experience for the patient. Hypotubes are also relatively low in cost.

Steerable mechanisms are also widely available for catheter delivery and navigation in a variety of minimally invasive surgical procedures. They may be used to deliver or push a catheter or other device, such as a pacemaker lead, through tortuous anatomy or to locate the entrance to an artery or vein.

Prior art steerable devices have often sacrificed many of the advantages of non-steerable delivery devices such as hypotubes. Typical steering mechanisms involve the use of a pull wire, which is connected to the distal end of the catheter tip. When a tensile force is applied to the pull wire, the tip of the catheter will bend proportionally in response to the force applied. Since the wire must be contained and controlled in some way, these steerable devices usually include a dedicated lumen to enclose the pull wire. This has the disadvantage of increasing manufacturing costs of the steerable device. It also has the disadvantage of adding weight to the product and increasing the overall profile of the device, thus making the procedure more uncomfortable for the patient. In addition, relatively complex handles are required in order to control tension in the pull wire. These are often bulky, with poor ergonomics and can be costly to manufacture. Furthermore, the pull wires are used under tension, and there is thus a possibility of breakage of the pull wires under tension during use.

U.S. Pat. No. 6,783,510 relates to a steerable catheter including a single pull wire arranged to allow the catheter to achieve various complex curvatures. The pull wire extends through two different offset lumens and attaches to the distal end of the catheter at an off-axis location. By tensioning the pull wire, the catheter can assume various complex curves, depending on the respective lumen through which the pull wire passes. A further disadvantage with this type of catheter is that the tip of the catheter may only be deflected in a single plane. This restriction limits the effectiveness of this type of catheter in reaching many of the desired treatment sites.

Steerable catheter devices often involve use of polymer extrusions. As described above, steerable systems that include pull wires require a separate lumen for each pull wire. Polymer extrusions are often used to create tubes with lumens as they may be mass-produced more cheaply than metallic tubes with lumens. Due to the fact that the material yield strength, and thus the column strength of a polymer tube is significantly lower than that of a steel tube, use of such extrusions is likely to result in a delivery device with lower pushability and torqueability than hypotubes. In an attempt to address this shortcoming, some manufacturers have used a braided polymer. A braided polymer comprises a metal braid embedded in a polymer jacket. However, the resulting device still does not perform as well as a metal hypotube. This type of arrangement may also lead to ‘springback’ in the device. Springback occurs when the inherent elasticity and memory of the polymer materials results in the polymer tube creeping away from a desired shaped or bent position towards its original straight position. This can make small, accurate movements difficult to achieve.

If a device were to provide perfect torque (or 1:1 torque), steerability would only be required in a single plane, as all other planes could be accessed by rotational displacement of the device. Due to decreased levels of torque provided by prior art steerable devices, some manufacturers have attempted to increase the steerability to compensate for lack of torqueability.

This may be done by introducing more pull-wires to steer in multiple planes, as described in U.S. Pat. No. 5,383,852. This relates to a steerable catheter with independent proximal and distal control. The catheter has an elongate flexible shaft and a flexible tip assembly connected to the distal end of the shaft. The tip assembly comprises a distal section and a proximal section coaxial with the shaft. The stiffness and length of the distal and proximal sections are selected to provide a predetermined curve configuration of the tip assembly when the distal section or proximal section is bent. The catheter includes two pairs of pull cables, which extend through the catheter. The first pair of cables is anchored at a first point in the tip assembly for bending the proximal section in a predetermined plane. The second pair of cables is anchored at a second point in the tip assembly for bending the distal section in a predetermined plane. This allows any desired orientation of the bending planes to be achieved. A handle or actuator is used to apply tension to the pull cables in order to control the deflection of the tip assembly. However, including additional pull wires in the assembly has the effect of compounding the original problems of large profile devices, high manufacturing costs and more complex handles.

Many of these devices sacrifice the working inner lumen in order to keep the diameter low. Since the capability to deliver a device or a treatment through the lumen is now lost, a second device may be necessary to carry out this function. This results in longer surgical procedures, increased material cost per procedure and increased risk to the patient. Another disadvantage of the loss of the working lumen is that the surgeon can no longer track the catheter over a guidewire, which is a widespread practice in percutaneous, minimally invasive procedures.

U.S. Pat. No. 6,802,835 relates to a steerable catheter device comprising an outer sheath, which engages a handle, and a flexible catheter with a shape memory tip which also engages the handle and which extends through the sheath. In a fully extended position, the memory tip extends at substantially 90 degrees to the longitudinal axis of the device. As the tip is withdrawn into the sheath from the fully extended position, the angle of the tip relative to the longitudinal axis can be varied infinitely between about 90 degrees and 0 degrees. When the tip is withdrawn so as to be entirely contained within the sheath, the tip is substantially aligned with the longitudinal axis of the catheter. One disadvantage of this steerable catheter is that longitudinal movement of one shaft relative to the other is required. This means that a device, such as a stent or balloon, loaded at the distal end of the catheter would prevent the necessary movement of the memory tip. For this reason, the catheter device may not be suitable for placing a stent or balloon within a body vessel of a patient. Furthermore, as the tip of the device is flexible, pushing a rigid device such as an ablation catheter through the inner member would be likely to force the flexible tip to straighten.

International Patent Publication No. WO2004/009150 relates to a telescopic introducer with a compound curvature for inducing alignment. The introducer comprises a flexible elongate outer sheath, and a flexible elongate telescopic inner sheath or core, telescopically disposed in the outer sheath. The outer and inner sheaths are rotatable relative to one another. A portion of the outer element has a first shape and a more proximal portion of the inner element also has the first shape. The inner element has a second shape on its distal portion. When the portions of the inner and outer elements having matching first shapes are aligned, the distal portion of the inner element will be extending in a predetermined selected orientation as intended for access to a desired portion of the coronary sinus branch venous system.

It is therefore desirable to provide a steerable catheter device, which includes the desirable properties of a hypotube delivery device, such as pushability, torqueability and kink resistance. It is also desirable to provide a steerable device which may be controlled more simply than prior art devices. It is also desirable to provide a steerable catheter device with a working inner lumen.

An object of the invention is to provide a steerable catheter, which includes the desirable properties of a hypotube delivery device such as pushability, trackability, kink resistance and torqueability. It is also an object to provide a steerable catheter with a working lumen. Another object is to provide a single use steerable catheter which is lightweight and inexpensive to manufacture.

### SUMMARY OF THE INVENTION

The present invention provides a steerable catheter assembly, comprising an elongate outer shaft having a lumen therethrough and having a proximal end and a distal end; an elongate inner shaft coaxially disposed within the lumen of the outer shaft and having a proximal end and a distal end; and characterised in that at least one of the inner shaft and the outer shaft is formed with a curvature at a distal portion, such that in an aligned configuration, a distal end of the catheter assembly is disposed at a maximum deflection angle α to a longitudinal axis of the catheter assembly; and the inner shaft and the outer shaft are rotatable relative to one another out of the aligned configuration such that one shaft exerts a deflection force on the other shaft to deflect the distal end of the catheter assembly between α and 0 degrees to the longitudinal axis of the catheter assembly.

In one embodiment of the invention, the inner shaft includes an inner lumen extending therethrough. In other embodiments, the inner shaft may comprise a solid bar or mandrel, which may be made from a flexible metal, shape memory metal or plastics material.

Preferably, the outer shaft containing the lumen has a high column strength. The outer shaft is typically metallic. Preferably, the outer shaft is formed from stainless steel. Alternatively, the outer shaft may be formed from nitinol, a cobalt based alloy, or a suitable polymer. Preferably, the inner shaft has a high column strength. The inner shaft is typically metallic. Preferably, the inner shaft is formed from stainless steel. Alternatively, the inner shaft may be formed from nitinol, a cobalt based alloy, or a suitable polymer. An advantage of the catheter assembly of the present invention is that it provides the desirable properties of a hypotube, such as pushability, trackability, torqueability and kink resistance, while also providing the steerability normally associated with more complex pull wire systems. Another advantage of the catheter assembly of the present invention is that a working inner lumen may be provided.

According to an embodiment of the invention, the inner shaft is formed with a curvature at a distal portion, and the outer shaft is formed with a curvature at a distal portion, and in the aligned configuration, the distal portion of the inner shaft is substantially aligned with the distal portion of the outer shaft, such that the distal end of the catheter assembly is disposed at a maximum deflection angle α to a longitudinal axis of the catheter assembly; and the inner shaft and the outer shaft are rotatable through an angle of rotation which may be in the range of 0° to about 180° relative to one another so that the distal portion of the inner shaft is moved out of alignment with the distal portion of the outer shaft, such that each shaft exerts a deflection force on the other shaft to deflect the distal end of the catheter assembly between α and 0 degrees to the longitudinal axis of the catheter assembly. In one embodiment the angle of relative rotation of the shafts is between 0° and about 45°, but more typically is between 0° and about 90°. Preferably the path of relative rotation lies in a single plane.

According to another embodiment of the invention, one shaft is formed with a curvature which lies in a first bend plane and the other, non-curved shaft is capable of bending in a second bend plane while resisting bending in other planes. When the shafts are in the aligned configuration, the first and second bend planes are aligned with one another and the entire assembly exhibits a maximum curvature at its distal end, corresponding to the curvature of the curved shaft. When the shafts are rotated relative to one another, the first and second bend planes are rotated out of alignment with one another. Since the non-curved shaft is formed to resist bending in planes other than the second bend plane, the deflection or curvature displayed at the distal end of the assembly is reduced.

According to an embodiment of the present invention, the inner shaft is formed with a curvature at a distal portion, and the distal portion of the outer shaft is capable of bending in a single pre-determined bend plane only, and in the aligned configuration, the curvature of the distal portion of the inner shaft is substantially aligned with the bend plane of the distal portion of the outer shaft, such that the distal end of the catheter assembly is disposed at a maximum deflection angle α to a longitudinal axis of the catheter assembly; and the inner shaft and the outer shaft are rotatable relative to one another so that the distal portion of the inner shaft is moved out of alignment with the bend plane of the distal portion of the outer shaft to deflect the distal end of the catheter assembly between α and 0 degrees to the longitudinal axis of the catheter assembly. The angle of rotation may be in the ranges stated above. Ideally, the outer shaft comprises a relatively rigid material, and a plurality of slots are formed in a portion of the wall of the outer shaft in a substantially spiral or circumferential path to allow a distal portion of the outer shaft to bend in a single pre-determined bend plane only.

According to a further embodiment of the present invention, the outer shaft is formed with a curvature at a distal portion, and the distal portion of the inner shaft is capable of bending in a single pre-determined bend plane only, and in the aligned configuration, the curvature of the distal portion of the outer shaft is substantially aligned with the bend plane of the distal portion of the inner shaft, such that the distal end of the catheter assembly is disposed at a maximum deflection angle α to a longitudinal axis of the catheter assembly; and the inner shaft and the outer shaft are rotatable relative to one another so that the distal portion of the outer shaft is moved out of alignment with the bend plane of the distal portion of the inner shaft to deflect the distal end of the catheter assembly between α and 0 degrees to the longitudinal axis of the catheter assembly. The angle of rotation may be in the ranges stated above. Ideally, the inner shaft comprises a relatively rigid material, and a plurality of slots are formed in a portion of the wall of the inner shaft in a substantially spiral or circumferential path to allow a distal portion of the inner shaft to bend in a single pre-determined bend plane only.

According to yet another embodiment of the present invention, the outer shaft and the inner shaft are displaceable longitudinally relative to one another out of the aligned configuration, such that each shaft exerts a deflection force on the other shaft to deflect the distal end of the catheter assembly between α and 0 degrees to the longitudinal axis of the catheter assembly.

In an embodiment of the invention, the distal portion of the inner shaft, or the distal portion of the outer shaft, or the distal portions of both shafts may comprise a shape memory material.

In another embodiment of the invention, the distal portion of the inner shaft, or the distal portion of the outer shaft or the distal portion of both shafts may comprise a shaped metal coil or spring.

According to yet another embodiment of the present invention, at least one spacer is disposed in the lumen of the outer shaft between the outer shaft and the inner shaft, such that an intermediate working lumen is established therebetween. An advantage of this arrangement is that a second working lumen is thus available for use during the surgical procedure. The second working lumen may be used for delivery of instruments to a site within the body, delivery of contrast media or for inflation of a balloon catheter. An additional advantage is that concentricity between the inner and outer shafts is maintained.

According to another aspect of the invention there is provided a steerable catheter assembly, comprising an elongate outer shaft having a lumen therethrough and having a proximal end and a distal end; an elongate inner shaft coaxially disposed within the lumen of the outer shaft and having a proximal end and a distal end; and characterised in that the distal portion of the inner shaft is capable of bending in a pre-determined bend plane only, and the distal portion of the outer shaft is capable of bending in a pre-determined bend plane only, and in an aligned configuration, the bend plane of the distal portion of the inner shaft is substantially aligned with the bend plane of the distal portion of the outer shaft, such that a distal end of the catheter assembly is capable of bending in the pre-determined bend plane with a maximum flexibility; and the inner shaft and the outer shaft are rotatable relative to one another out of the aligned configuration so that the bend plane of the distal portion of the inner shaft is moved out of alignment with the bend plane of the distal portion of the outer shaft to vary the flexibility of the distal end of the catheter assembly between the maximum flexibility and a minimum flexibility.

In one embodiment, a plurality of slots are formed in a portion of the wall of the outer shaft in a substantially spiral or circumferential path to allow a distal portion of the outer shaft to bend in a pre-determined bend plane only. A plurality of slots may be formed in a portion of the wall of the inner shaft in a substantially spiral or circumferential path to allow a distal portion of the inner shaft to bend in a pre-determined bend plane only.

An advantage of this arrangement is that when inserting a device in a body lumen of a patient, such as for example, a pacemaker lead, is that the catheter assembly may be stiffened when it is required to push the device through a blockage, and may be made more flexible when it is required to navigate through the tortuous pathways of the vasculature. Prior art systems require separate stiff and flexible styluses to be used, wherein each stylus is alternately inserted and withdrawn from the body as required.

Several embodiments of the steerable catheter assembly of the present invention will now be described with reference to and/or as illustrated in the accompanying drawings.

### BRIEF DESCRIPTION OF THE DRAWINGS

FIG. 1 is a side elevation of a first embodiment of the steerable catheter of the present invention;

FIG. 2 is a side elevation of the steerable catheter assembly of FIG. 1, in which the inner shaft has been rotated relative to the outer shaft;

FIG. 3 is a perspective view of one embodiment of an inner shaft of the steerable catheter assembly of the present invention;

FIG. 4 is a longitudinal cross section of the steerable catheter assembly of FIG. 1;

FIG. 5 is a longitudinal cross section of a further embodiment of the steerable catheter assembly of the present invention;

FIG. 6A is a side elevation of a fourth embodiment of the steerable catheter of the present invention;

FIG. 6B is a perspective view of the steerable catheter of FIG. 6A;

FIG. 7A is a plan view of a portion of a shaft according to one embodiment of the present invention, in a laid flat state to facilitate understanding of the configuration of the slots;

FIG. 7B is a perspective view of the portion of FIG. 7A;

FIG. 8A is a side elevation of a one embodiment of the steerable catheter assembly of the present invention (in the straightened position);

FIG. 8B is a perspective view of the steerable catheter assembly of FIG. 8A;

FIG. 9 is a side elevation of a second embodiment of the steerable catheter assembly according to the present invention;

FIG. 10 is a side elevation of a third embodiment of the steerable catheter assembly of the present invention;

FIGS. 12A and 12B are side elevations of a further embodiment of a steerable catheter assembly of the present invention;

FIGS. 13 and 13A are side elevations of a further embodiment of a steerable catheter assembly of the present invention;

FIG. 14 is a side elevation of a further embodiment of the steerable catheter assembly of the present invention;

FIG. 15A is a perspective view of a steerable catheter assembly according to a second aspect of the invention, in an aligned position;

FIG. 15B is a perspective view of the steerable catheter assembly of FIG. 15A, moved out of the aligned position; and

FIG. 16 is a side elevation of a shaft for use in an embodiment of the steerable catheter assembly according to the present invention.

### DETAILED DESCRIPTION OF PREFERRED EMBODIMENT

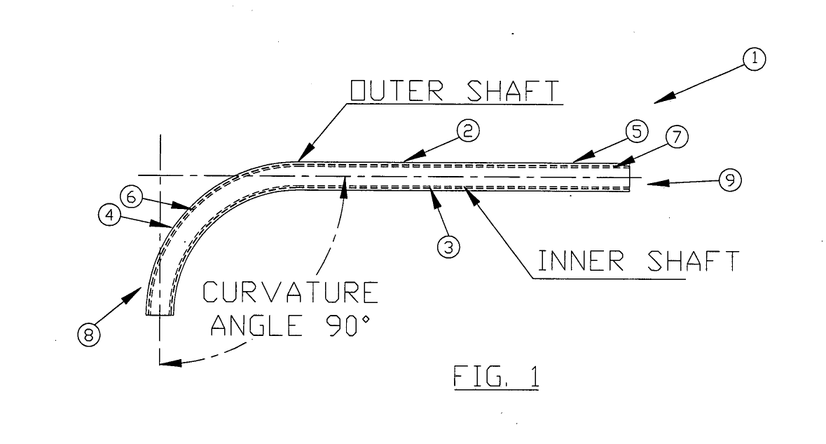

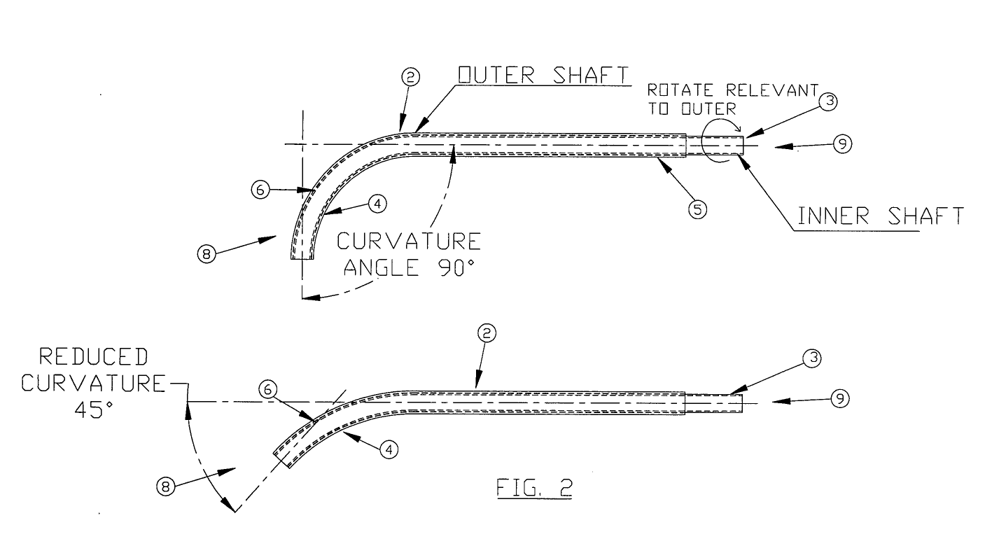

A first embodiment of the steerable catheter assembly of the present invention is shown in FIGS. 1 and 2. The catheter assembly 1 comprises a stainless steel outer shaft 2, having a lumen extending therethrough and a stainless steel inner shaft 3, having a lumen extending therethrough. Each of the shafts is of thin-walled construction. In alternative embodiments, the shafts may be formed from other metals, or from suitable plastics. The inner shaft is coaxially disposed within the outer shaft.

The outer shaft 2 is formed with a bend (or curvature) of approximately 90 degrees at a distal portion 4. The bend or curvature may be formed, for example, by heat treatment of the shaft. In an ‘at rest’ or unstressed position, the outer shaft distal end 4 will be disposed at an angle of approximately 90 degrees to the proximal end 5 of the shaft. The bend or curvature is such that if the outer shaft is manually straightened, the shaft will automatically return to its unstressed position on release. The amount of curvature in the distal end 4 of the outer shaft 2 in the unstressed position (90 degrees in the present embodiment) represents the maximum steering angle of the catheter assembly 1.

Similarly, the inner shaft 3 is formed with a bend (or curvature) of approximately 90 degrees at a distal portion 6. In an “at rest” or unstressed position, the inner shaft distal end 6 will be disposed at an angle of approximately 90 degrees to the proximal end 7 of the shaft. The bend or curvature is such that if the inner shaft 3 is manually straightened, the shaft will automatically return to its unstressed position on release.

In a first configuration shown in FIG. 1, the distal portion 4 of the outer shaft 2 is substantially aligned with the distal portion 6 of the inner shaft 3, such that the catheter assembly 1 has a curvature of approximately 90 degrees at its distal end 8. In the present embodiment, the catheter assembly may be adjusted between angles of 0 degrees and 90 degrees. In alternative embodiments, other angles of curvature may be used.

As shown in FIG. 2, the steering mechanism works by effecting relative rotational displacement between the inner and outer shafts. In the at rest position shown in FIG. 1, before any rotation takes place the inner shaft 3 and outer shaft 2 are aligned and the catheter assembly distal end 8 is disposed at an angle to the assembly proximal end 9. This angle is the ‘at rest’ angle formed by the shaft distal ends with the shaft proximal ends. In the embodiment shown in FIGS. 1 and 2, the outer shaft 2 is clamped at its proximal end 5 so that it cannot rotate. The inner shaft 3 is freely rotatable within the outer shaft. If the inner shaft is rotated, the bend at the inner shaft distal portion 6 is no longer aligned with the bend at the outer shaft distal portion 4. This results in a force being exerted on the outer shaft distal end 4 by the inner shaft distal end 6, which causes the outer shaft distal end 4 (and thus the catheter assembly distal end 8) to begin to straighten. If the inner shaft 3 is rotated, for example, through about 180°, the inner shaft distal portion 6 will be fully misaligned with the outer shaft distal portion 4, such that the curvature in the inner shaft distal end 6 is disposed in an opposite direction to that in the outer shaft distal end 4. The forces exerted by each shaft on the other are equal and opposite and the catheter assembly is caused to straighten. By rotating the inner shaft relative to the outer different angles of curvature (between 90 and 0 degrees) of the catheter assembly distal end 8 may be achieved. In alternative embodiments, the outer shaft is rotated while the inner shaft is held in place, or each shaft may be rotated by up to about 90 degrees in opposite directions.

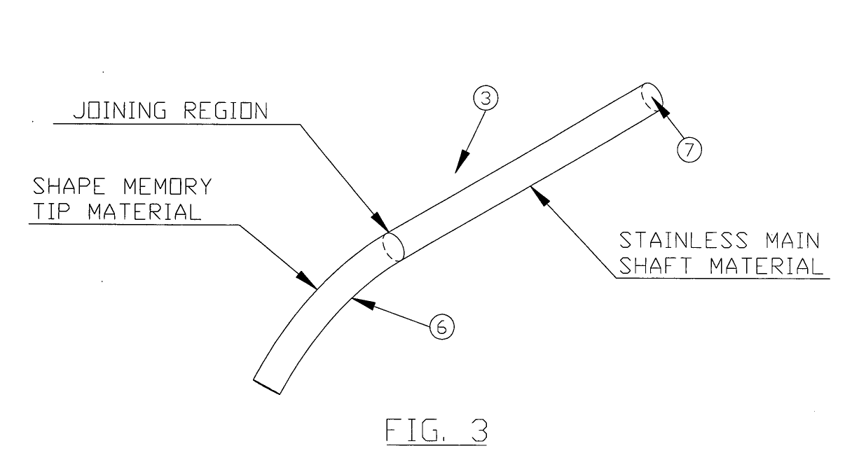

In the embodiment shown in FIG. 3, the distal portion 6 of the inner shaft 3 comprises a shape memory material (such as nitinol). Nickel-based, copper-based, iron-based, platinum-based or polymer shape memory materials may also be used. Shape memory materials offer excellent elasticity, but may be relatively expensive. For this reason, the shape memory material is used only for the distal portion 6 of the inner shaft 3. The shape memory section is attached to the proximal shaft using a mechanical fit, a weld or other known joining method. The catheter assembly operates as described above. In alternative embodiments, the distal portion of the outer shaft is formed from a shape memory material, or the distal portions of both the inner and outer shafts are formed from a shape memory material.

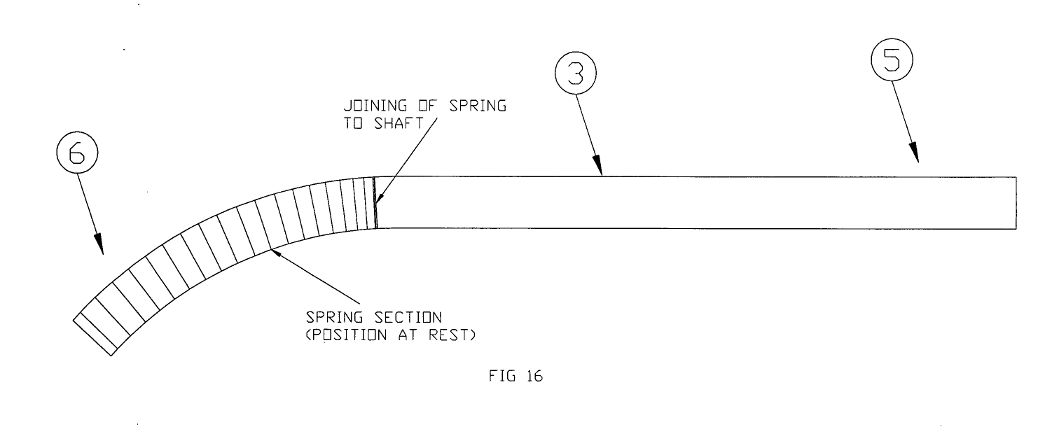

An alternative shaft for use in an embodiment of the present invention is shown in FIG. 16. In this embodiment, the distal portion 6 of the inner shaft 3 (or of the outer shaft 2 or of both the inner and outer shafts) comprises a metal coil. This provides a cheaper alternative to the shape memory material. The metal coil is similar to a spring and may be formed with a curvature as previously described. The metal coil is attached to the proximal shaft by a mechanical fit, a weld or other known joining method.

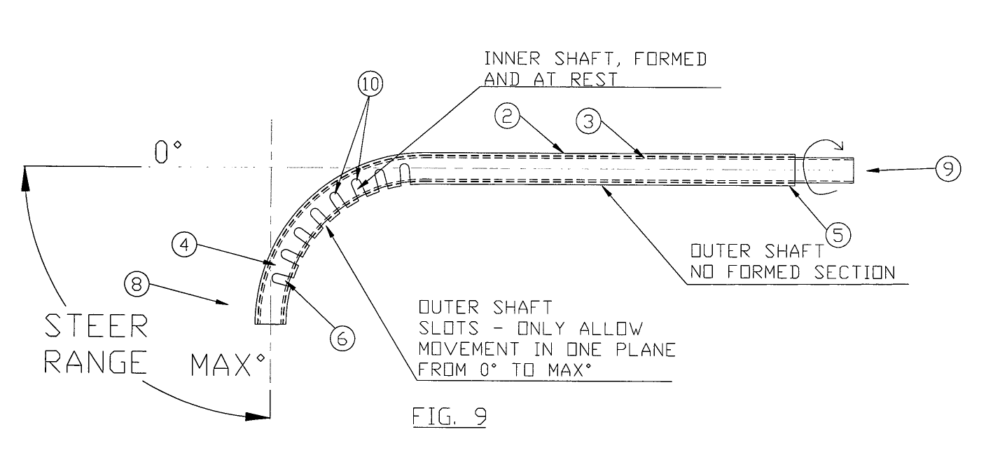

A second embodiment of the steerable catheter assembly of the present invention is shown in FIG. 9. The inner shaft 3 is formed with a curvature of approximately 90 degrees at a distal end 6. This curvature may be formed, for example, by heat treatment of the shaft 3 or by use of shape memory material as described above with reference to FIG. 3. The outer shaft 2 is not pre-formed with a bend or curvature but has had material removed at a distal portion 4 to form slots 10 on one side of the shaft 2. The outer shaft is formed from a material such as stainless steel and the slots 10 allow the outer shaft 2 to bend in one direction only. The slots render the shaft 2 sufficiently flexible to allow it to adopt the same shape as the inner shaft 3. In the ‘at rest’ position, the catheter assembly distal end 8 is therefore disposed at an angle of approximately 90 degrees to the catheter assembly proximal end 9. In this embodiment, the outer shaft 2 is clamped at its proximal end 5 so that it cannot rotate, whereas the inner shaft 3 is freely rotatable. When the inner shaft is rotated relative to the outer shaft, the outer shaft cannot deflect in any plane other than the single direction permitted by the slots, and the outer shaft thus begins to straighten. If the inner shaft is rotated through about 90 degrees, it exerts a deflection force on the outer shaft in the direction of curvature of the inner shaft. However, the rigidity of the outer shaft ensures that the outer shaft cannot be deflected (in any plane other than that allowed by the slots) beyond a straight position.

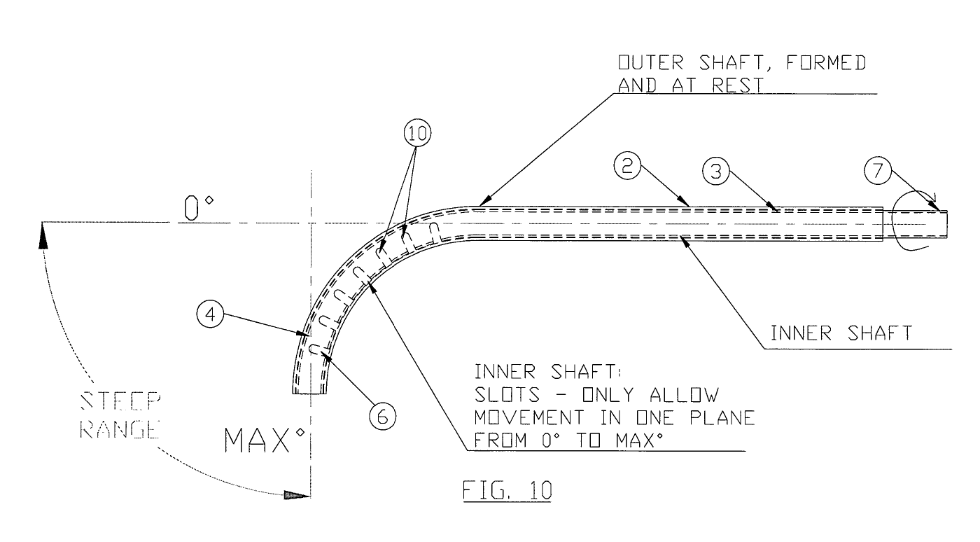

A third embodiment is shown in FIG. 10. In this embodiment, the outer shaft 2 is formed with a curvature of approximately 90 degrees at its distal end 4 as described above with reference to FIGS. 1 to 3. The inner shaft 3 is not formed with a bend or curvature but has had material removed at a distal portion 4 to form slots 10 on one side of the shaft 2. The inner shaft 3 is formed from a material such as stainless steel and the slots 10 allow the inner shaft 3 to bend in one direction only. The slots render the shaft 3 sufficiently flexible to allow it to adopt the same shape as the outer shaft 2. In the ‘at rest’ position, the catheter assembly distal end 8 is therefore disposed at an angle of approximately 90 degrees to the catheter assembly proximal end 9. In this embodiment, the inner shaft 3 is clamped at its proximal end 7 so that it cannot rotate, whereas the outer shaft 2 is freely rotatable. When the outer shaft is rotated relative to the inner shaft, the inner shaft cannot deflect in any plane other than the single direction permitted by the slots, and the inner shaft thus begins to straighten. If the outer shaft is rotated through about 90° degrees, it exerts a deflection force on the inner shaft in the direction of curvature of the outer shaft. However, the rigidity of the inner shaft ensures that the inner shaft cannot be deflected (in any plane other than that allowed by the slots) beyond a straight position.

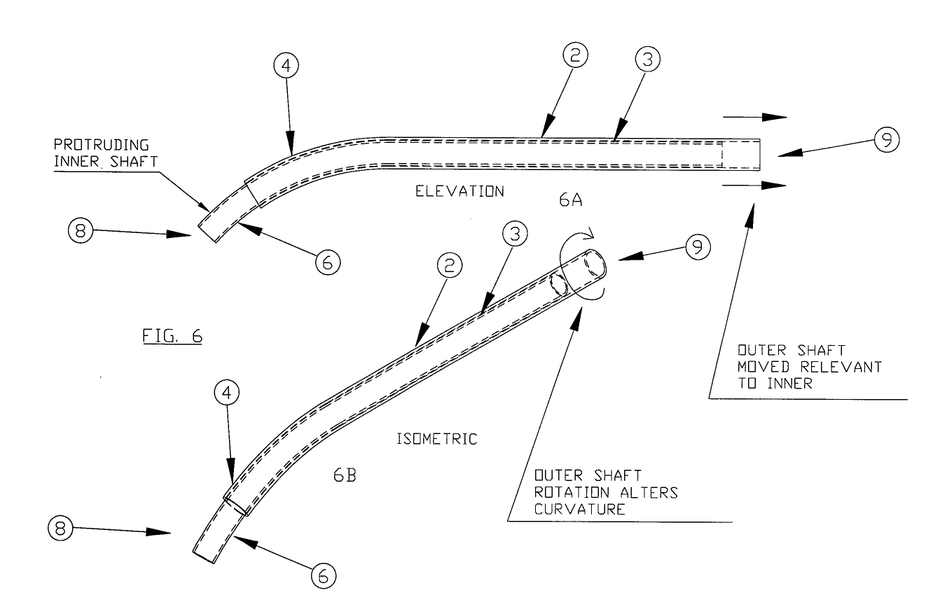

A fourth embodiment of the steerable catheter assembly 1 of the present invention is shown in FIGS. 6A and 6B. In this embodiment, both the inner and outer shafts 3, 2 are formed with a bend or curvature of approximately 90 degrees at their distal ends 6, 4 as previously described with reference to FIGS. 1 to 3. In the ‘at rest’ position, the distal end 8 of the catheter assembly 1 is disposed at approximately 90 degrees to the proximal end 9 of the assembly. The shape of the curvature of the assembly may be varied by effecting relative longitudinal movement between the inner and outer shafts as shown in FIG. 6A. Longitudinal displacement may be combined with relative rotational displacement as described above.

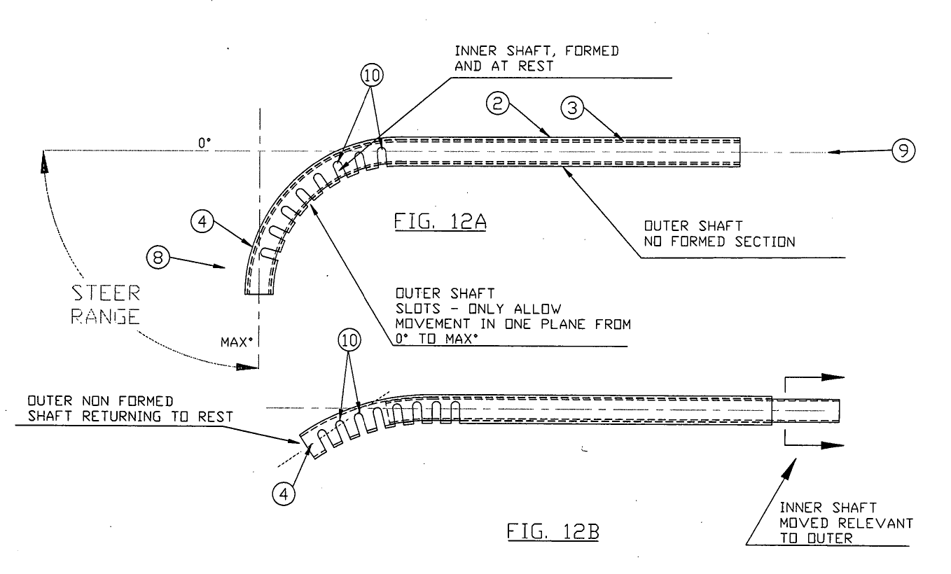

A fifth embodiment of the steerable catheter assembly 1 is shown in FIG. 12. In this embodiment, the inner shaft 3 is formed with a curvature of approximately 90 degrees at a distal end 6 as described above with reference to FIGS. 1 to 3. The outer shaft 2 is not pre-formed with a bend or curvature but has had material removed at a distal portion 4 to form slots 10 on one side of the shaft 2, as described above with reference to FIG. 9. In the ‘at rest’ position, the catheter assembly distal end 8 is therefore disposed at an angle of approximately 90 degrees to the catheter assembly proximal end 9. If the inner shaft is moved longitudinally relative to the outer shaft, in a proximal direction, the distal end 4 of the outer shaft 2 will begin to straighten as the deflection force exerted on it by the inner shaft is removed.

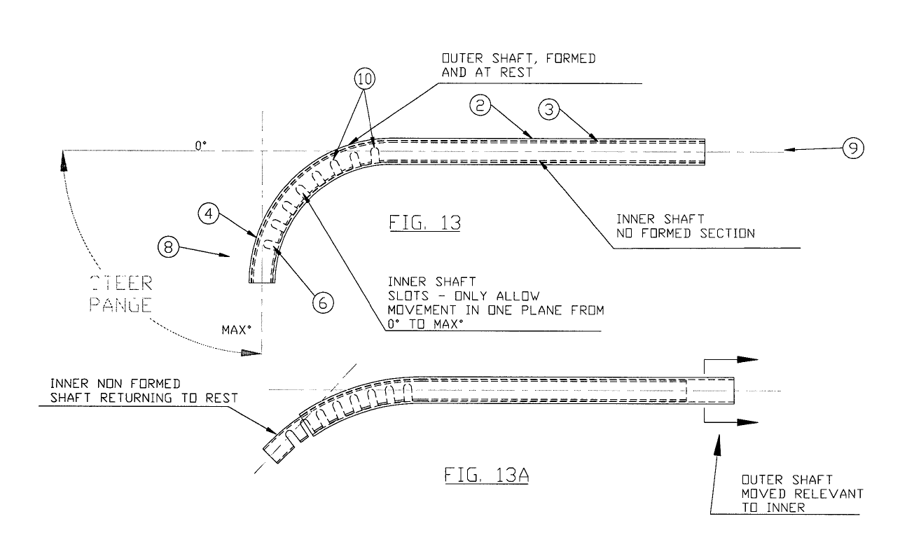

A further embodiment is shown in FIGS. 13 and 13A, in which the outer shaft 2 is formed with a curvature of approximately 90 degrees at its distal end 4 as described above with reference to FIGS. 1 to 3. The inner shaft 3 is not formed with a bend or curvature but has had material removed at a distal portion 4 to form slots 10 on one side of the shaft 2 as described above with reference to FIG. 10. In the ‘at rest’ position, the catheter assembly distal end 8 is therefore disposed at an angle of approximately 90 degrees to the catheter assembly proximal end 9. If the outer shaft is moved longitudinally relative to the inner shaft, in a proximal direction, the distal end 6 of the inner shaft 3 will begin to straighten as the deflection force exerted on it by the outer shaft is removed.

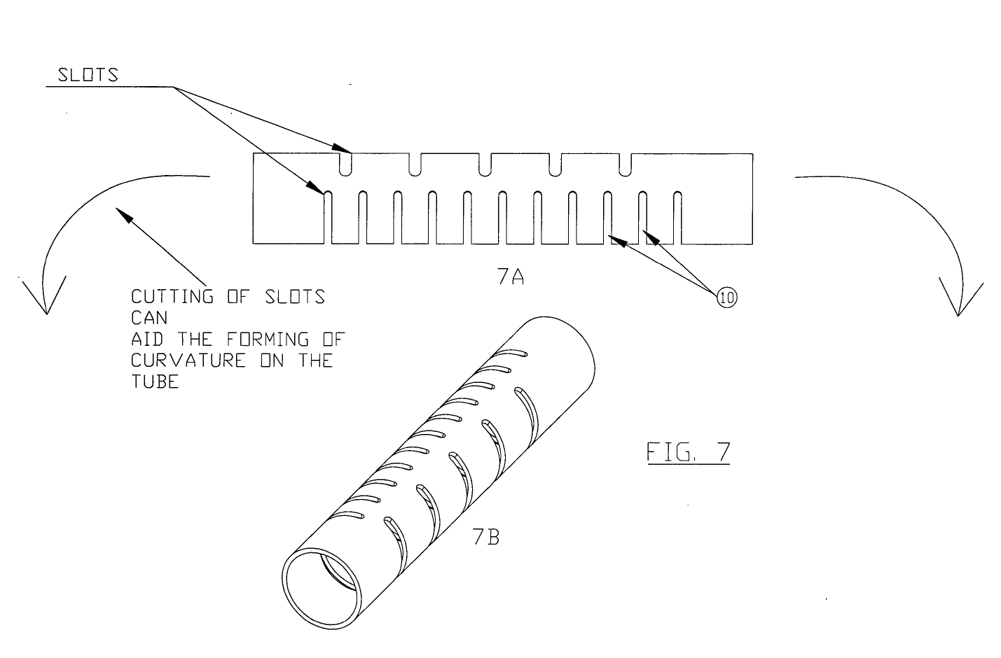

In a further embodiment of the present invention, as shown in FIGS. 7A and 7B, slots may be cut into the distal end of the inner shaft, the outer shaft or both shafts, in order to allow the shaft to bend or curve in a pre-determined direction. As described above with reference to FIGS. 9 and 10, slotting may be used to ensure that the shaft is only capable of bending in a single plane or direction. As shown in FIG. 7, slots may be cut into both sides of the shaft in order to ensure that the shaft bends in a pre-determined bend plane.

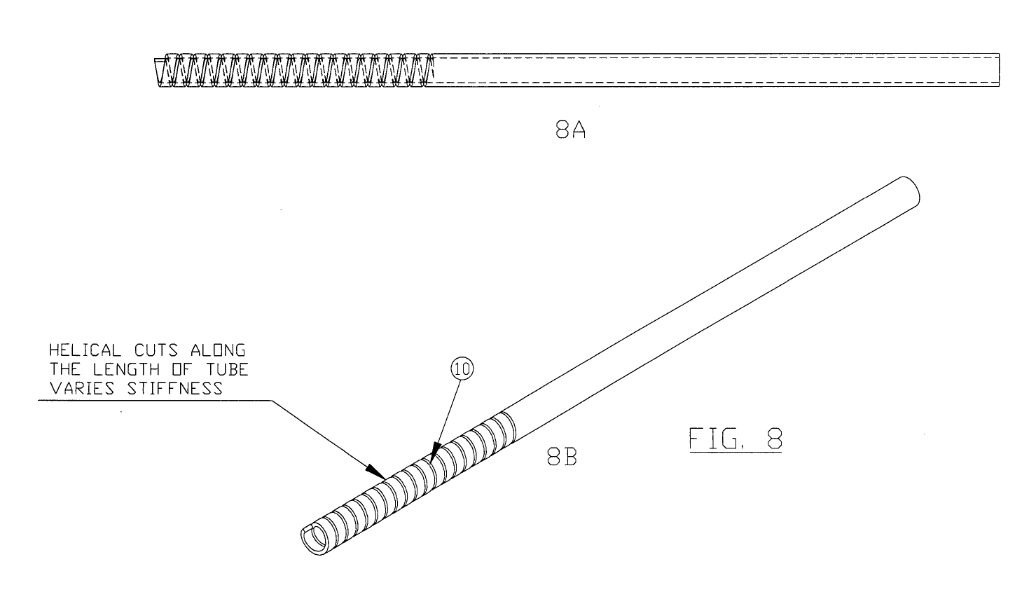

Slots may also be used to vary the stiffness of the shafts. For example, a spiral (or helical) slot may be cut into the wall of the shaft as shown in FIGS. 8A and 8B. The pitch of the spiral determines the degree of flexibility imparted to the shaft.

In general, as indicated above, the relative rotation of the shafts can be through an angle of rotation in the range 0° and about 180°, but typically through about 90°. In embodiments where both shafts are formed with a curvature (of about 90°), the catheter assembly will be substantially straight when the shafts have been rotated through about 180° relative to one another. When the shafts have been rotated through about 90° relative to one another, the distal end of the catheter assembly will be disposed at an angle of approximately 45° to the proximal end thereof. However, in embodiments where slots are formed in one shaft, the catheter assembly will be substantially straight when the shafts have been rotated through about 90° relative to one another. When the shafts have been rotated through about 45° relative to one another the distal end of the catheter assembly will be disposed at an angle of approximately 45° to the proximal end thereof. Table A below sets out the angle of curvature of the catheter assembly for various embodiments of the present invention.

TABLE A

| | | Relative rotation from aligned | |
|---|---|---|---|
| | | configuration | |
| Inner Shaft | Outer Shaft | 90° | 180° |
| Formed with 90° | Formed with 90° | Catheter | Catheter assembly |
| curvature at | curvature at | assembly | substantially |
| distal end | distal end | distal end | straight |
| | | disposed | |
| | | at 45° to | |
| | | proximal end | |
| Formed with 90° | Not formed with | Catheter | Catheter assembly |
| curvature at | curvature, but | assembly | disposed at 90° |
| distal end | formed with slots | substantially | in opposite |
| | to allow bending | straight | direction to aligned |
| | in one plane | | configuration |
| Not formed with | Formed with 90° | Catheter | Catheter assembly |
| curvature, but | curvature at | assembly | disposed at 90° |
| formed with slots | distal end | substantially | in opposite |
| to allow bending | | straight | direction to aligned |
| in one plane | | | configuration |
| Not formed with | Not formed with | Catheter | Catheter assembly |
| curvature, but | curvature, but | assembly | substantially |
| formed with slots | formed with slots | substantially | straight, |
| to allow bending | to allow bending | straight, | flexibility |
| in one plane | in one plane | flexibility | maximised |
| | | minimised | |

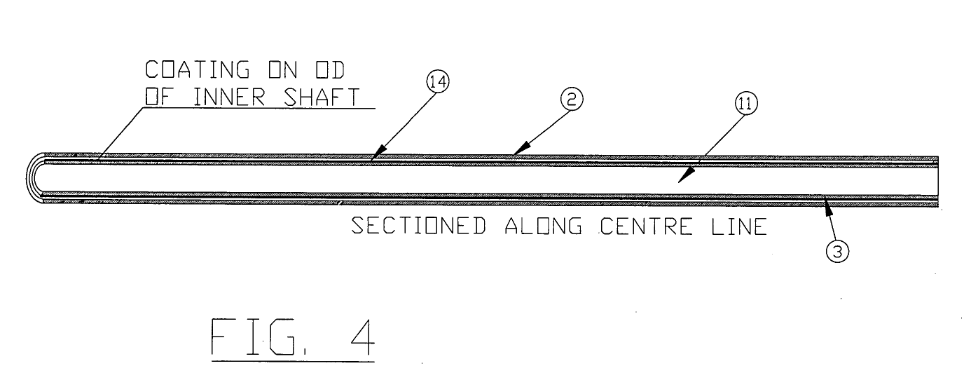

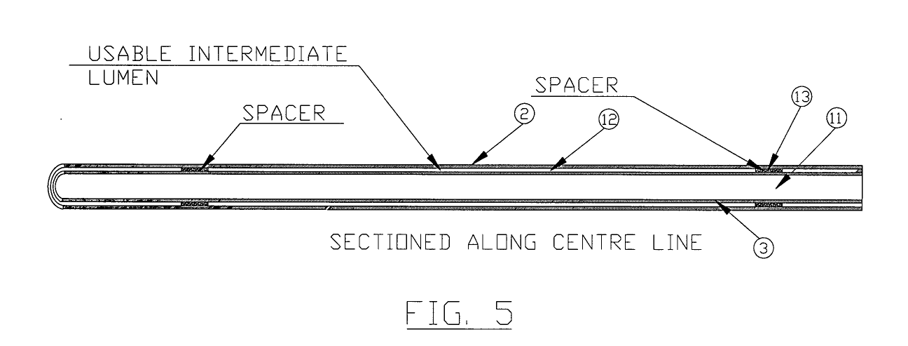

Referring now to FIGS. 4 and 5, it will be appreciated by those skilled in the art that an important advantage of the steerable catheter assembly of the present invention is that a working lumen 11 may be provided within the inner shaft of the catheter assembly. The inner working lumen 11 may be used, for example, to track over a guidewire, to inject contrast media, to deliver an endoscope to a site within the body or to supply inflation fluid to a catheter balloon. The working lumen may also be used for a variety of other applications.

In one embodiment of the present invention, as shown in FIG. 5, an intermediate lumen 12 is provided between the inner and outer shafts 3,2. Spacers 13 are positioned between the inner and outer shafts to provide an intermediate working lumen 12. In the embodiment shown, the spacers 13 are substantially annular, but are not continuous around the entire circumference of the inner shaft. In an alternative embodiment, the spacers 13 are formed with through-holes (in a direction substantially parallel to the longitudinal axis of the catheter assembly). This ensures that fluid and/or devices may pass through the intermediate lumen. The wall thickness of the spacers 13 determines the size of the intermediate lumen 12. The spacers are pushed onto the outer wall of the inner shaft 2 before assembly of the steerable catheter. The spacer has the additional advantage of maintaining concentricity between the inner and outer shafts. The intermediate working lumen 12 may be used for any of the applications described above with reference to the inner working lumen 11. Additionally, in conjunction with the inner working lumen, the catheter assembly can be used, for example, to simultaneously track and deliver contrast media.

As shown in FIG. 4 of the drawings, the inner shaft 3 may be provided with a coating 14. The coating serves to reduce frictional forces between the inner and outer shafts. In the embodiment shown in FIG. 4, a coating 14 is applied to the outer wall of the inner shaft 2. However, in alternative embodiments, the coating 14 may be applied to the inner wall of the outer shaft 2 or to both shafts. The coating may comprise, for example, an extruded polymer overjacket, a heat shrink polymer overjacket, a dipped coating, a sprayed coating or any type of coating known in the art. The coating 14 may be hydrophobic or hydrophilic in nature, depending on the end application of the catheter assembly. In the embodiments shown in FIGS. 7 to 10, where slots are formed in one or both shafts, a polymer overjacket coating may be used to seal the lumen within the shaft, so that it becomes fluid tight.

In all of the embodiments described above, a handle is provided to control the steering mechanism of the catheter assembly. The handle is connected to the inner and outer shafts, such that relative rotation between the shafts may be effected. In certain embodiments, the handle is also arranged to allow relative longitudinal displacement between the inner and outer shafts.

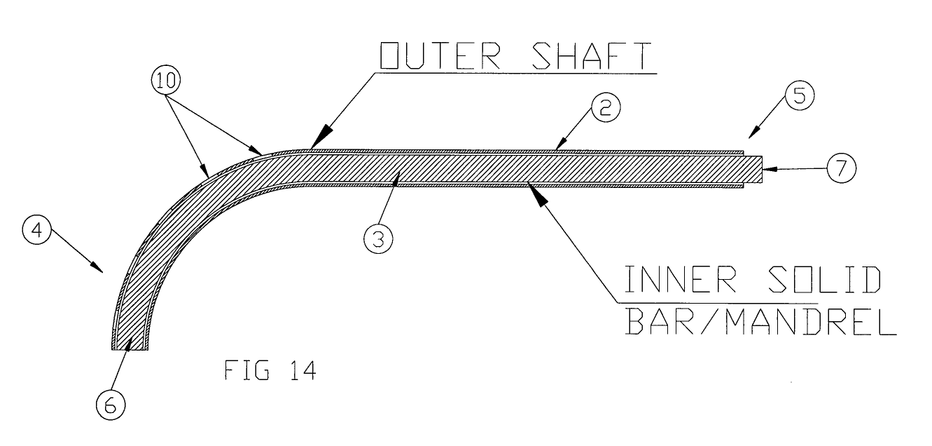

Referring now to FIG. 14 of the drawings, there is provided a steerable catheter assembly 1, comprising an elongate outer shaft 2 having a lumen therethrough and having a proximal end 5 and a distal end 4; an elongate inner shaft 3 coaxially disposed within the lumen of the outer shaft and having a proximal end 7 and a distal end 6. The inner shaft 3 comprises a flexible solid bar or mandrel and is formed with a curvature at a distal portion 6 as described above. The outer shaft 2 is not pre-formed with a bend or curvature but has had material removed at a distal portion 4 to form slots 10 in the shaft 2, as described above with reference to FIG. 9. The slots may be formed in one or alternatively both sides of the shaft. Suitably, the solid bar is made from a shaped memory metal, such as nitinol. Nickle-based, copper-based, iron-based, platinum-based or polymer shape memory materials may also be used.

In a first configuration shown in FIG. 14, the distal portion 4 of the outer shaft 2 is substantially aligned with the distal portion 6 of the inner shaft 3, such that the catheter assembly 1 has a curvature of approximately 90 degrees at its distal end 8. In the present embodiment, the catheter assembly may be adjusted between angles of 0 degrees and 90 degrees. In alternative embodiments, other angles of curvature may be used.

As previously described, the steering mechanism works by effecting relative rotational displacement between the inner and outer shafts. In the ‘at rest’ position, the catheter assembly distal end 8 is therefore disposed at an angle of approximately 90 degrees to the catheter assembly proximal end 9. In this embodiment, the outer shaft 2 is clamped at its proximal end 5 so that it cannot rotate, whereas the inner shaft 3 is freely rotatable. When the inner shaft is rotated relative to the outer shaft, the outer shaft cannot deflect in any plane other than the single direction permitted by the slots, and the outer shaft thus begins to straighten. If the inner shaft is rotated through about 90° degrees, it exerts a deflection force on the outer shaft in the direction of curvature of the inner shaft. However, the rigidity of the outer shaft ensures that the outer shaft cannot be deflected (in any plane other than that allowed by the slots) beyond a straight position.

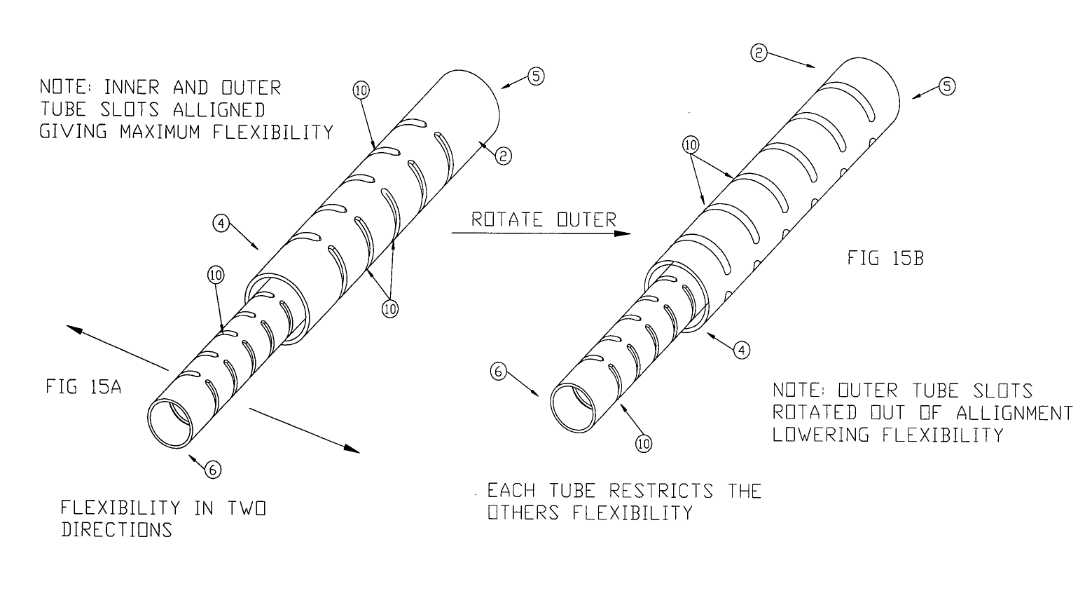

Referring now to FIG. 15A and FIG. 15B of the drawings, there is provided a steerable catheter assembly according to a second aspect of the invention, comprising an elongate outer shaft 2 having a lumen therethrough and having a proximal end 5 and a distal end 4 and an elongate inner shaft 3 coaxially disposed within the lumen of the outer shaft and having a proximal end 7 and a distal end 6. The distal portion 6 of the inner shaft 3 is capable of bending in a pre-determined bend plane only, and the distal portion 4 of the outer shaft 2 is capable of bending in a pre-determined bend plane only. The outer and inner shafts have had material removed at a distal portion to form slots 10, as described above with reference to FIGS. 9 and 10. The slots may be formed in one side of the shaft or more typically in two opposite side of the shafts. In alternative embodiments, other slot configurations may be used.

In an aligned configuration as shown in FIG. 15A, the bend plane of the distal portion of the inner shaft is substantially aligned with the bend plane of the distal portion of the outer shaft, such that a distal end of the catheter assembly is capable of bending in the pre-determined bend plane with a maximum flexibility. As shown in FIG. 15B, the inner shaft and the outer shaft are rotatable relative to one another out of the aligned configuration so that the bend plane of the distal portion of the inner shaft is moved out of alignment with the bend plane of the distal portion of the outer shaft to vary the flexibility of the distal end of the catheter assembly between the maximum flexibility and a minimum flexibility. Thus, rotating one shaft relative to the other results in a stiffening of the catheter assembly.

The words “comprises/comprising” and the words “having/including” when used herein with reference to the present invention are used to specify the presence of stated features, integers, steps or components but does not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

1. A steerable catheter assembly, comprising:
an elongate outer shaft having a lumen therethrough and having a proximal end and a distal end;
an elongate inner shaft coaxially disposed within the lumen of the outer shaft and having a proximal end and a distal end; and
characterised in that at least one of the inner shaft and the outer shaft is formed with a curvature at a distal portion, such that in an aligned configuration, a distal end of the catheter assembly is disposed at a maximum deflection angle α to a longitudinal axis of the catheter assembly; and
the inner shaft and the outer shaft are rotatable relative to one another out of the aligned configuration such that each shaft exerts a deflection force on the other shaft to deflect the distal end of the catheter assembly between α and 0 degrees to the longitudinal axis of the catheter assembly.

2. A steerable catheter assembly as claimed in claim 1, characterised in that the inner shaft includes an inner lumen extending therethrough.

3. A steerable catheter assembly as claimed in claim 1, characterised in that:
the inner shaft is formed with a curvature at a distal portion, and the outer shaft is formed with a curvature at a distal portion, and in the aligned configuration, the distal portion of the inner shaft is substantially aligned with the distal portion of the outer shaft, such that the distal end of the catheter assembly is disposed at a maximum deflection angle α to a longitudinal axis of the catheter assembly; and
the inner shaft and the outer shaft are rotatable through an angle of rotation which may be in the range of 0° and about 180° relative to one another so that the distal portion of the inner shaft is moved out of alignment with the distal portion of the outer shaft, such that each shaft exerts a deflection force on the other shaft to deflect the distal end of the catheter assembly between α and 0 degrees to the longitudinal axis of the catheter assembly.

4. A steerable catheter assembly as claimed in claim 1, characterised in that:
the inner shaft is formed with a curvature at a distal portion, and the distal portion of the outer shaft is capable of bending in a pre-determined bend plane only, and in the aligned configuration, the curvature of the distal portion of the inner shaft is substantially aligned with the bend plane of the distal portion of the outer shaft, such that the distal end of the catheter assembly is disposed at a maximum deflection angle a to a longitudinal axis of the catheter assembly; and
the inner shaft and the outer shaft are rotatable between 0 and 90 degrees relative to one another so that the distal portion of the inner shaft is moved out of alignment with the bend plane of the distal portion of the outer shaft to deflect the distal end of the catheter assembly between α and 0 degrees to the longitudinal axis of the catheter assembly.

5. A steerable catheter assembly as claimed in claim 4, characterised in that a plurality of slots are formed in a portion of the wall of the outer shaft in a substantially spiral or circumferential path to allow a distal portion of the outer shaft to bend in a pre-determined bend plane only.

6. A steerable catheter assembly as claimed in claim 1, characterised in that:
the outer shaft is formed with a curvature at a distal portion, and the distal portion of the inner shaft is capable of bending in a pre-determined bend plane only, and in the aligned configuration, the curvature of the distal portion of the outer shaft is substantially aligned with the bend plane of the distal portion of the inner shaft, such that the distal end of the catheter assembly is disposed at a maximum deflection angle a to a longitudinal axis of the catheter assembly; and
the inner shaft and the outer shaft are rotatable between 0 and 90 degrees relative to one another so that the distal portion of the outer shaft is moved out of alignment with the bend plane of the distal portion of the inner shaft to deflect the distal end of the catheter assembly between α and 0 degrees to the longitudinal axis of the catheter assembly.

7. A steerable catheter assembly as claimed in claim 6, characterised in that a plurality of slots are formed in a portion of the wall of the inner shaft in a substantially spiral or circumferential path to allow a distal portion of the inner shaft to bend in a pre-determined bend plane only.

8. A steerable catheter assembly as claimed in claim 1, further characterised in that the outer shaft and the inner shaft are displaceable longitudinally relative to one another out of the aligned configuration, such that each shaft exerts a deflection force on the other shaft to deflect the distal end of the catheter assembly between α and 0 degrees to the longitudinal axis of the catheter assembly.

9. A steerable catheter assembly as claimed in claim 1, wherein the outer shaft is formed from stainless steel.

10. A steerable catheter assembly as claimed in claim 1, wherein the inner shaft is formed from stainless steel.

11. A steerable catheter assembly as claimed in claim 1, characterised in that the distal portion of the inner shaft, or the distal portion of the outer shaft, or the distal portions of both shafts comprises a shape memory material.

12. A steerable catheter assembly as claimed in claim 1, characterised in that the distal portion of the inner shaft, or the distal portion of the outer shaft or the distal portion of both shafts comprises a metal coil.

13. A steerable catheter assembly as claimed in claim 1, characterised in that at least one spacer is disposed in the lumen of the outer shaft between the outer shaft and the inner shaft such that an intermediate working lumen is established therebetween.

14. A steerable catheter assembly, comprising:
an elongate outer shaft having a lumen therethrough and having a proximal end and a distal end;
an elongate inner shaft coaxially disposed within the lumen of the outer shaft and having a proximal end and a distal end; and
characterised in that the distal portion of the inner shaft is capable of bending in at least one pre-determined bend plane only, and the distal portion of the outer shaft is capable of bending in at least one pre-determined bend plane only, and in an aligned configuration, the at least one bend plane of the distal portion of the inner shaft is substantially aligned with the at least one bend plane of the distal portion of the outer shaft, such that a distal end of the catheter assembly is capable of bending in the pre-determined bend plane with a maximum flexibility; and
the inner shaft and the outer shaft are rotatable relative to one another out of the aligned configuration so that the at least one bend plane of the distal portion of the inner shaft is moved out of alignment with the at least one bend plane of the distal portion of the outer shaft to vary the flexibility of the distal end of the catheter assembly between the maximum flexibility and a minimum flexibility.

15. A steerable catheter assembly as claimed in claim 14, characterised in that a plurality of slots are formed in a portion of the wall of the outer shaft in a substantially spiral or circumferential path to allow a distal portion of the outer shaft to bend in a pre-determined bend plane only.

16. A steerable catheter assembly as claimed in claim 14, characterised in that a plurality of slots are formed in a portion of the wall of the inner shaft in a substantially spiral or circumferential path to allow a distal portion of the inner shaft to bend in a pre-determined bend plane only.
